(19) United States
(12) Patent Application Publication
(10) Pub. No.: US 2014/0242175 A1
(43) Pub. Date: Aug. 28, 2014

(54) NANO-ENCAPSULATED, CONTROLLED DRUG DELIVERY, MANUFACTURING PROCESS AND SYSTEM

(71) Applicant: Albert Solur Zeng, Cupertino, CA (US)

(72) Inventor: Albert Solur Zeng, Cupertino, CA (US)

(21) Appl. No.: 13/775,016

(22) Filed: Feb. 22, 2013

(51) Int. Cl. A61K 9/51; A61K 9/00

(52) U.S. Cl. CPC A61K 9/51; A61K 9/5192; A61K 9/0002
USPC 424/490; 427/2.14

(57) ABSTRACT

A mixed dose of a nanosized drug wherein at least one portion of the mixed dose comprises a core nanosized drug encapsulated in at least one layer of a protective material having the same core drug or different core drug. A mixed dose of a nanosized drug wherein at least one portion of the mixed dose comprises a core nanosized drug encapsulated in at least one shell of a protective material with same drug concentration or different drug concentrations. A mixed dose of a nanosized drug wherein at least one portion of the mixed dose comprises a core nanosized drug encapsulated such that to have different release schedule than the other portions of the drug. Methods and systems for the manufacturing and the administration of nanosized encapsulated drugs are also provided.

### BACKGROUND OF THE INVENTION

1. Field of the Invention

The invention relates generally to nanobiopharmaceutics and more particularly to nano-encapsulated drugs, their controlled and/or scheduled delivery method, manufacturing process and system for processing nano size, delivery controlled encapsulated drugs.

2. Description of the Related Art

Traditional medicine administered orally may have a slower and less complete absorption than medicine administered using parenteral (non-oral) routes. Dissolution of solid formulations (e.g., tablets) must occur first. The drug must survive exposure to stomach acid and this route of administration is subject to the first pass effect (metabolism of a significant amount of drug in the gut wall and the liver), before it reaches the systemic circulation where it can take effect.

Even if it reaches the systemic circulation, the route of the drug is completely random. It may flow around and be expelled from the body without performing its job.

Because it is hard for the drug to find its desired target, a lot of the drug is wasted, and a large amount of the medicine must be administrated, increasing toxicity in the body and causing unnecessary medicine waste. More damaging is that, by circulating throughout the body looking for a target, and by increasing the toxicity level of the body, these traditional medicines kill both, good cells and bad cells.

In addition, the traditional drugs/medicines are expelled out of the body in a very short time period, which is why some medicines need to be taken multiple times a day for several days. An example is Amoxicillin, which may need to be taken every 6 hours per day, 7 days per treatment session.

In summary, traditional drugs have low effectiveness and efficiency, they may require repeated administration, they cause high levels of body toxicity, and they are expensive.

One of the challenges of the pharmaceutical research nowadays is to discover tools and methods enabling an effective and efficacious delivery of drugs into the tissues or organs where the drugs are needed, and in addition, scheduling delivery of the drugs in a controlled manner.

Nanomedical approaches to drug delivery center on developing nanoscale particles or molecules to improve drug bioavailability. Bioavailability refers to the presence of drug molecules where they are needed in the body, where they will do the most good, and over a period of time desired. More than $65 billion are wasted each year due to poor bioavailability of existing drugs. Thus, drug delivery research focuses on maximizing bioavailability both at specific places in the body and over a period of time.

Protein and peptides exert multiple biological actions in human body and they have been identified as showing great promise for treatment of various diseases and disorders. These macromolecules are called biopharmaceuticals. Targeted and/or controlled delivery of these biopharmaceuticals using nanomaterials like nanoparticles and dendrimers is an emerging field called nanobiopharmaceutics, and these products are called nanobiopharmaceuticals.

Two forms of nanomedicine that have already been tested in mice and are apparently awaiting human trials is using gold nanoshells to help diagnose and treat cancer, and using liposomes as vaccine adjuvants and as vehicles for drug transport.

It has been seen that drug detoxification is also another application for nanomedicine which has shown promising results in rats. A benefit of using nanoscale for medical technologies is that smaller devices are less invasive and can possibly be implanted inside the body. In addition, biochemical reaction times are much shorter. These devices are faster and more sensitive than typical drug delivery.

This strategy took the fashionable name of ‘nanomedicine’ (medical application of nanotechnology), mainly based on the use of lipid-based (liposomes) and polymer-based (nanoparticles; NPs) nanocarriers or metalbased nanovectors. The last example of nanocarriers (i.e., superparamagnetic NPs) is currently used in medicine in order to improve the quality and the specificity of body/cell imaging and diagnostics. These carriers are usually made of gold or iron, comprising a core-shell able to be visualized within the body, thus allowing the physician to obtain better-defined contrast and diagnostic images.

(http://www.futuremedicine.com/doi/pdf/10.2217/nnm.12.90).

Nano encapsulated drugs are nano sized packages of drugs that are encapsulated/covered with layer(s) such as liposomes and/or of polymer or other bio degradable protective materials, that protect the drugs inside (core drugs) from unfavorable environments and prevent the drug from taking effect until the capsule dissolves. The cover or the coating can delay the drug release. Liposomes and other lipid-based nanocapsules cannot be applied to many other drugs. Other than liposomes, no other nanocapsules are known to be available due to the difficulties of manufacturing them.

Thus, there is a need for the development of new medicine, namely nano-sized encapsulated medicines, capable of providing time controlled delivery, and which are easier to administer to target area, require fewer administrations, have lower toxicity, and are less expensive overall. Furthermore, since making nano-sized encapsulated medicine is very challenging, there is also a need for providing processes, procedures, and systems to make nano-sized encapsulated medicines possible.

The problems and the associated solutions presented in this section could be or could have been pursued, but they are not necessarily approaches that have been previously conceived or pursued. Therefore, unless otherwise indicated, it should not be assumed that any of the approaches presented in this section qualify as prior art merely by virtue of their presence in this section of the application.

### BRIEF SUMMARY OF THE INVENTION

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In one exemplary embodiment, a system for the manufacturing of nanosize encapsulated drugs is provided. In another exemplary embodiment, a process for the manufacturing of nanosize encapsulated drugs is provided. In another exemplary embodiment, nanosize encapsulated drugs having different protective layers in terms of number of layers, layer thickness and material used, are provided. In another exemplary embodiment, mixed layered encapsulated drug with same core drug or different core drugs, and having the same or different concentrations, are provided.

Thus, it is now possible to efficiently and effectively manufacture nanosize encapsulated drugs capable of providing time controlled delivery with the same or different core drugs and which are easier to administer to target area, require fewer administrations, have lower toxicity, and are less expensive overall.

The above embodiments and advantages, as well as other embodiments and advantages, will become apparent from the ensuing description and accompanying drawings.

### BRIEF DESCRIPTION OF THE DRAWINGS

For exemplification purposes, and not for limitation purposes, embodiments of the invention are illustrated in the figures of the accompanying drawings, in which:

FIG. 1 illustrates sectional views of non-coated nanosize medicine, and coated nanosize medicine having one, two and three protective layers, according to several embodiments.

FIG. 2 illustrates sectional views of doses of non-coated nanosize medicine and mixed nanosize encapsulated medicine, according to one embodiment.

FIG. 3 illustrates sectional views of a wafer having nanosize dents, at different stages of the manufacturing process of nanosize encapsulated drugs, according to several embodiments.

FIG. 4 illustrates the side view of an exemplary system for the manufacturing of nanosize encapsulated drugs, according to an embodiment.

FIG. 5 is a flow chart depicting an exemplary process for the manufacturing of nanosize encapsulated drugs, according to an embodiment.

### DETAILED DESCRIPTION OF THE PREFERRED EMBODIMENTS

What follows is a detailed description of the preferred embodiments of the invention in which the invention may be practiced. Reference will be made to the attached drawings, and the information included in the drawings is part of this detailed description. The specific preferred embodiments of the invention, which will be described herein, are presented for exemplification purposes, and not for limitation purposes. It should be understood that structural and/or logical modifications could be made by someone of ordinary skills in the art without departing from the scope of the invention. Therefore, the scope of the invention is defined by the accompanying claims and their equivalents.

For the purpose of this disclosure, the protective material that constitutes the coating layer(s) for the core nanodrug may be a polymer or any other suitable biodegradable material.

For the purpose of this disclosure, the configuration(s) of the material that constitutes the coating layer(s) for the core nanodrug means physical and/or chemical construction, physical and/or chemical formations, physical staggering, and so on.

As stated earlier, nano encapsulated drugs are nano sized packages of drugs that are encapsulated/covered with layer(s) such as liposomes and/or of polymer or other biodegradable protective materials, that protect the drugs inside (core drugs) from unfavorable environments and prevent the drug from taking effect until the capsule dissolves. The cover or the coating can delay the drug release. More importantly by mixing such coated nano drugs, as it will be described herein, the total half life of the drug may be increased.

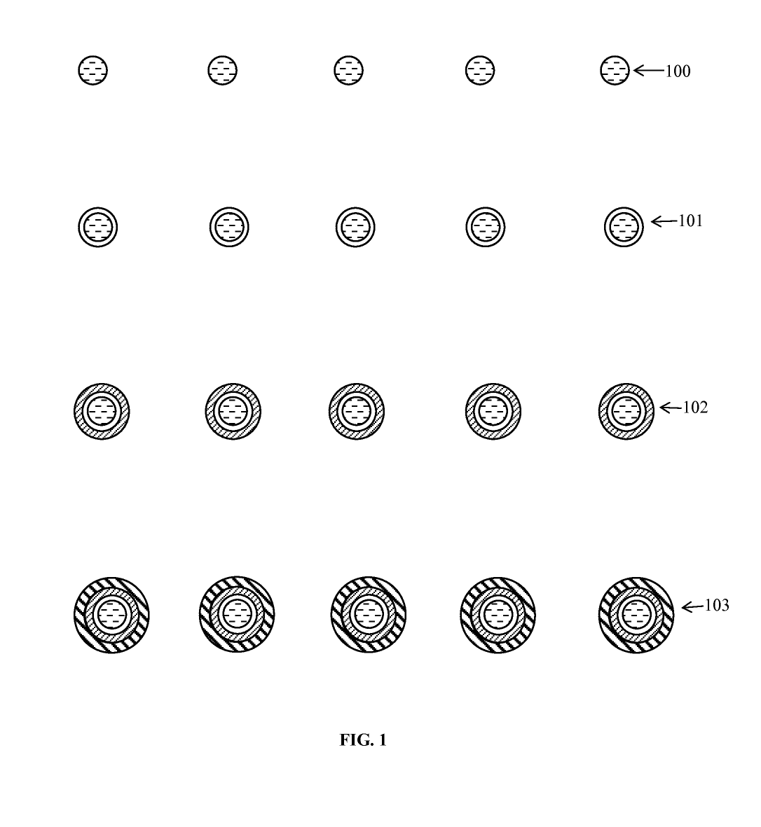

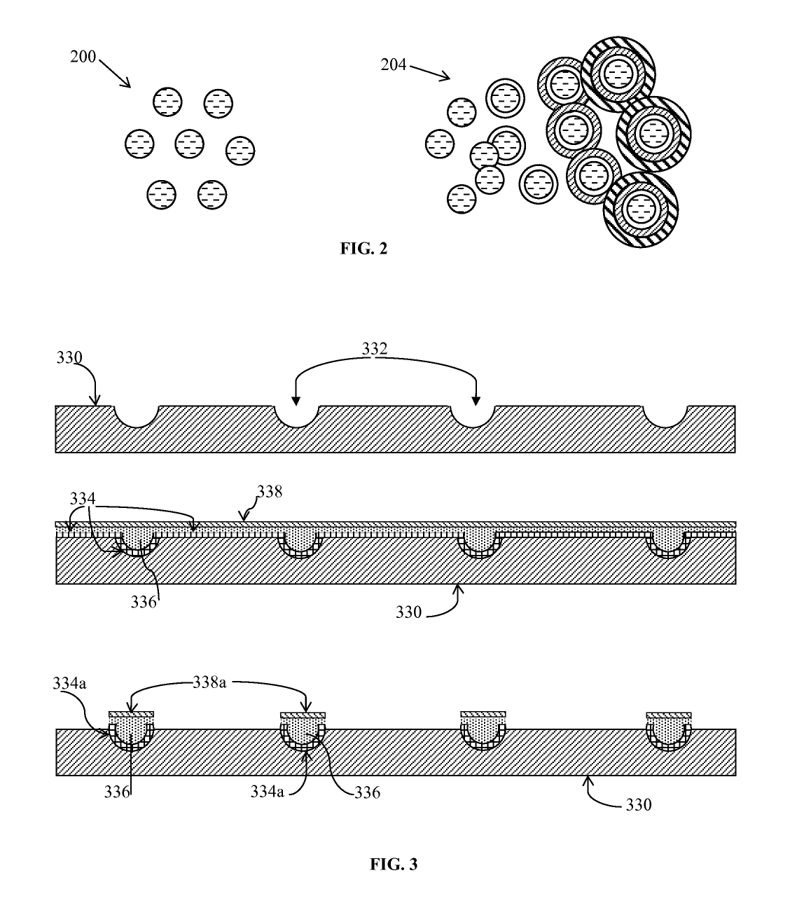

Referring now to FIG. 1, a group of non-coated nanosize drug particles 100 is shown, together with groups of nanosize encapsulated drugs having one 101, two 102 or three 103 protective layers, according to several embodiments. It should be apparent that more than three protective layers may be used for the purposes described herein. In FIG. 2, what is shown is a dose of non-coated nanosize drugs 200 and a mixed dose 204 of nanodrugs including non-coated, one-layer, two-layer and three-layer nanodrugs. The mixed dose 204, according to an embodiment, may have nanocapsules with protective layers which are different in terms of protective layer number, thickness and material used. Thus, after mixing, longer, scheduled delivery of the drug may be obtained by using protective layers as it will be explained in more details hereafter. Having a portion of non-coated nanosize drugs in a mixed dose 204 may be preferred in most situations to ensure immediate drug action by the non-coated portion. However, the mixed dose 204 may contain only coated nanosize drugs when the purpose is to delay any drug action (e.g., by one week) after the administration of the mixed dose.

Here are some exemplary scenarios that may help understand the inventions disclosed herein:

First scenario: Let's assume a dose of a specific nanodrug contains nanocapsules having a single shell/bilayer liposome and an X (e.g., 4-5 nm) nanometer thick polymer layer. The nanodrug is administered to the patient. For the purpose of this example, let's assume that its half life time inside the human body is about one week. The drug will stay inside the human body for about three weeks.

Second scenario: If the polymer thickness of the one-bilayer liposome nanocapsules is doubled to 2X nanometers and such nanocapsules are combined/mixed with nanocapsules having a single bilayer liposome and a single X nm polymer layer, the expected half life time of the mixed drug can be around two weeks. And, the mixed drug can be expected to last inside of the body around six weeks. Nano capsulation of the polymers in this case may have to be compressed, meshed, smashed, or nested to certain physical formation or configuration to achieve protection of the Liposomes to prevent the Liposome to dissolve or to prevent core drug to leak out before schedule time frame.

The above scenarios, given as examples, show that the half life time and the presence-duration or release time of nanodrugs can be increased as desired, by increasing the number of protective layers, and/or the thickness of the protective layer(s), and/or by selecting a suitable coating material for the protective layer(s), and/or by suitably altering the physical configuration, structure or construction of the protective layer(s) of the nanocapsules such as by smashing, meshing, compressing or nesting techniques. Thus, to time control the presence/release time and the half life time of a specific nanodrug dose administered into human body, the dose may have mixed half life time nanodrugs, with different release schedule.

Mixed nanocapsules, having various number of layers and/or layer thickness, and/or layer material, and/or physical configuration of the protective layers will control when to release the drug, will allow to mix or combine different release times of the drugs, will increase total half life time of the drug, and also provide the possibility of varying drug delivery concentration at specific periods of time. For example, if, when the mixed nanodose is initially prepared and administered, it is anticipated that during the second week of the treatment, more concentrated drug(s) will be needed in the body or in the ill tissue in order to achieve effective treatment given the known behavior of the disease, the concentration of the nanodrug(s) scheduled for release during the second week may be increased in the mixed nanodose. Thus, a mixed nanodose 204 (FIG. 2) may have various components (e.g., non-coated, one-layer, two-layer nanodrug, etc) in equal or different concentrations, with the same, different or a combination of core nanodrug in each component, depending on the delivery schedule desired and/or the treatment objectives.

Mixed nano-capsules, having various layer materials, number of layers and/or layer thickness, and/or physical configuration of the protective layers will thus control when to release the drug, will allow to mix or combine different release time of the drugs, will increase total half life time of the same drug, and also provide the possibility of varying drugs and delivery at specific periods of time. For example, if, after a surgery, first time period needs to stop bleeding, second time period needs to control the possible infections, and third time period needs to cure left over un-cleared tumors, but there is also the need to strengthen the patient by adding nutrition all the time, then mixed drugs can be scheduled such that during first time period the drug to stop bleeding is released, during the second time period the drug to control the possible infections is released, and during the third time period the drug to cure left over un-cleared tumors is released, while nutrition is released all the time.

Thus, besides the nature (material used), the number of layers and/or the thickness of the protective layers (e.g., polymer, or other bio-degradable material), other factors will control the half life time and the release time or duration of the presence (presence-duration) of the nanodrug in the human body. Examples of such factors are the doping material used if any, or the composition and/or the physical structure of the protective layer(s). All of these factors together will determine when, where and how the nanocapsule will dissolve and when the drug will take effect. Thus, controlling these factors during the manufacturing of the nanodrugs will translate into time control of the nanodrug delivery.

The scheduled drug delivery may be further understood by using the example that follows. Let's assume that a nanodrug A contains three different categories/types of nanodrugs, A1, A2 and A3, in terms of number of layers, thickness and/or nature of the protective material used for the protective layer(s), and/or protective layer(s) physical configuration. The differences in thicknesses, layer numbers of protective layers and/or materials used, and/or material configurations cause the different nanocapsules to have different release times. For example, if drug A1 releases during the first week, drug A2 releases during the second week and drug A3 releases during the third week, mixed together, the different release times result in a continuous release time over a period of three weeks.

If the above example is extended to “n” drugs, and thus, if drugs A1, A2, A3, A4, A5, . . . . An have been mixed together and administrated, theoretically, the resulting release time of the drug can be about “n” weeks.

What follows are considerations regarding the processes and systems for the manufacturing of nano-sized encapsulated drugs.

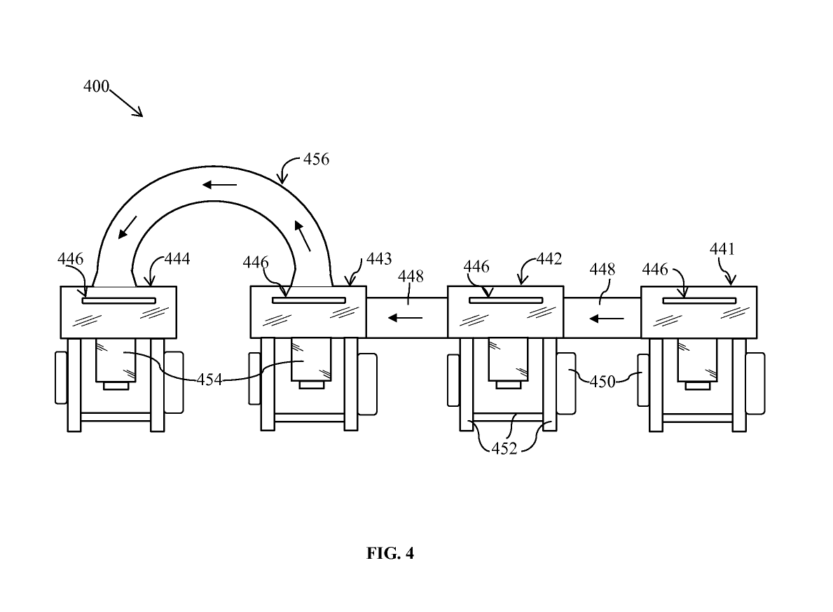

When the core drugs (i.e., the drug to be encapsulated) have nano-sized solid particle or crystals already available, then only encapsulation (one or more layers) of the nano-sized drugs is needed using preferably only the fourth chamber (tumbler) 444 in FIG. 4 as it will be explained below. If the core drugs are in liquid form, nano filtering membrane processes are needed to form nano sized core drugs. And again, only fourth chamber 444 will preferably be used to coat the nanosized core drugs obtained through filtering.

If the core drugs are in molecular or gas forms or even in solid/dust, or crystal form, the below processes may be performed to shape up the nano drugs and to obtain the first layer of encapsulation (may not be completely enclosed), and thus, a solid shell nanosized drug capsule.

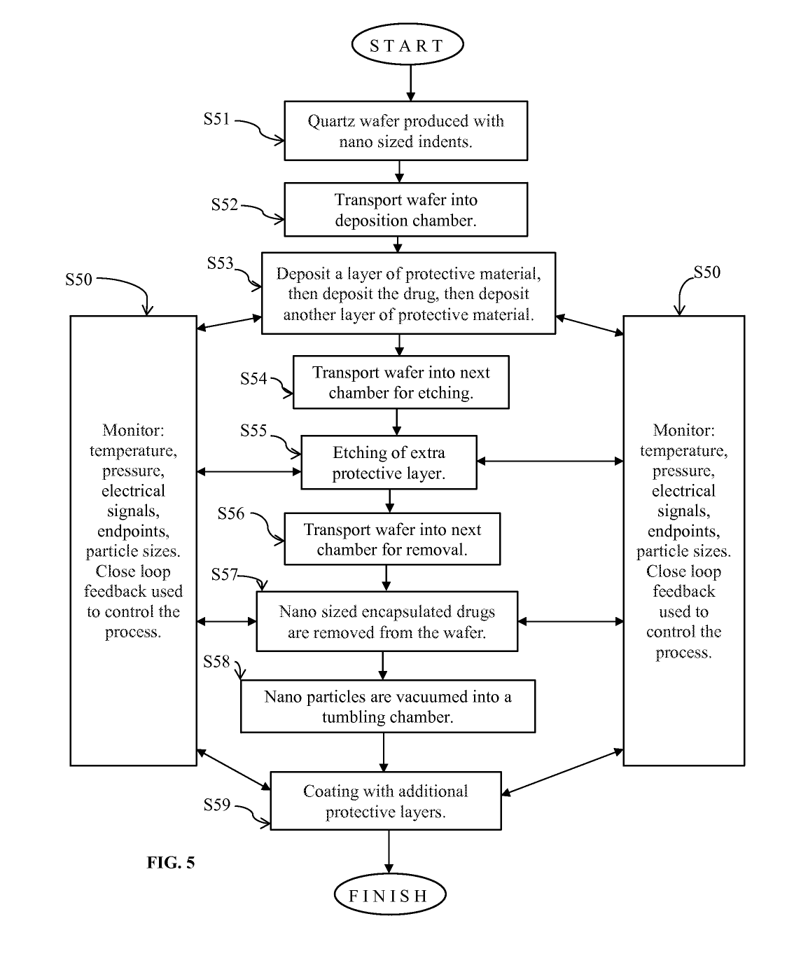

First (i.e., Step S51, FIG. 5), typically, wafers 330 (FIG. 3) made from glass, quartz or other suitable materials and having nano-sized indents/cavities/dents 332 are produced. The dents' size is to be determined by the drug's nanosize needed. For example, the dents' size may be as small as 20 nm. Furthermore, the shape of the dents 332 may preferably be semispherical to allow easy removal of the nanosize drug from the dents.

Next, the indented wafer 330 is transported (Step S52, FIG. 5) into first chamber (deposition chamber) 441 (FIG. 4). A static electrode chuck (not shown) may be used to clamp the wafer 330 inside deposition chamber 441.

Next (Step S53, FIG. 5), protective material such as biodegradable polymer, is supplied or formed into the deposition chamber 441 to form a first/bottom layer 334 of protective material that coats the wafer 330 including its dents 332 with a, for example, 5 nm thick coat 334. The polymer particles may be caused to be attracted toward the wafer 330 by an electrical field.

Next, (Step S53, FIG. 5), the nanosized core drug 336 (FIG. 3) in gas, molecular or other form, is supplied to chamber 441 to fill the dents 332. The nanosized core drug in gas, molecular or other form may be supplied to chamber 441 together with other necessary agent(s) to fill the dents 332. During the deposition processes, chemical reaction and/or physical reaction may occur to form binding, form layers, or changing the gas to a non-gas form and/or form the small particles to the particle size of dents 332. These processes may be similar to the semiconductor deposition processes. It should be noted that, as shown in FIG. 3, the supplied nanodrug will typically also form a layer over the entire surface of the wafer 330, on top of first layer 334 of protective material.

Next, still in Step S53, FIG. 5, protective material is supplied again into the deposition chamber 441 to form another (second/top) layer 338 of protective material to cover the core drugs 336. Typically, the entire wafer 330 will also be covered with the second layer 338 of protective material. Thus, the excess protective material and core drug resting on the wafer 330, outside of the area occupied by the dents 332, will have to be removed.

Next, (Step S54, FIG. 5), using transport module 448 (FIG. 4) wafer 330, containing the core drug 336 and the two layers (334, 338) of protective material as described above, is transported into the second/etching chamber 442 for removal of excess protective material and core drug. Before etching, pattern photoresist technique may be used to differentiate the locations to be etched away or to be kept. In the etching chamber 442, (Step S55, FIG. 5) the excessive areas are removed by selective etching method so as to leave a layer of protective material (334a and 338a, FIG. 3) mostly wrapped around the core drug 336a and all no dent areas to be cleared.

Next, (Step S56, FIG. 5), after the etching process, the wafer 330 is transported, using transport module 448 into the third/removal chamber 443 where the nanosized encapsulated drugs are removed (Step S57) from the wafer 330. The removal may be accomplished by, for example, using vacuum (Step S58) to draw the wrapped nanodrugs, through the transport passage 456, into the fourth/tumbling chamber 444. For example, the use of temperature differences can be applied to cause the wafer dents to expand and the nano encapsulated drugs to contract, thus, allowing the drugs to be easily moved off the wafer, namely from the dents 332. 3D vibration can also be used to separate the nano encapsulated drugs from the wafer.

Thus, in chambers 441-443, the objective is to obtain a nanosize capsule from a drug that only exists in molecular or gas form. However, the process as described above in relation to chambers 441-443 may also be used to encapsulate nanodrugs available in other forms (e.g., solid small nanosize drug particles).

In the fourth chamber 444, (Step S59, FIG. 5), the nanosized drug capsules, already having one coat of protective material as explained above, may be coated with additional layers (one or more) of protective material. To accomplish this, in fourth chamber 444, the nanosized drug capsules may be supported by air, oxygen or any other bio allowable gas. The pressure of the supporting gas may be designed such that it can balance gravity by particle sizes, weight, and so on. Similarly, nanosized drug particles already existing in solid or crystal form may be processed directly in fourth chamber 444 (i.e., skipping chambers 441-443) for coating them with one more protective layers.

To coat the nanocapsules or the already solid or crystal nanoparticles with the additional protective layers, encapsulation materials are supplied into the fourth chamber 444 together with the supporting materials, and rotational and helical motions of the nanoparticles are caused by the fourth chamber (tumbler) 444 to close opened capsules, and to achieve uniform encapsulation. And, in chamber 444 process conditions may be changed as needed to compress, smash, meshes, and or nests the bio-degradable coating material to construct certain physical formation or configuration such that core drugs are to be protected and drug release time can be controlled.

It should be noted that during the entire encapsulation process, control systems 450 are used for each of the four chambers of the encapsulation system 400. Temperature controls, pressure controls, electrical signal controls, and so on, are designed into the system. Signal feedback loops are in place to control the encapsulation process (Step S50, FIG. 5).

Nano scale scopes are installed on the viewports 446 so the process can be monitored. Furthermore, as shown in FIG. 4, each chamber may be mounted on a frame 452 and each chamber may be equipped with a pump 454, or blower. Pump 454 will typically be used to create vacuum inside the chambers in order to create process condition needed and to draw the nanosized particles inside the chambers.

Online in-situ measurements and detection system are available.

The length of time the core drugs stay in the tumbler chamber are calculated/tested based on such factors as the release time needed, the protective material used, the physical configuration desired and/or the processing parameters (e.g., pressure, temperature, tumbling speed, possible layer configurations etc), which are controlled. For example, a first polymer (or other protective material) may be supplied to chamber 444 in which the temperature, pressure, tumbling speed and time are set particularly for this first polymer. Next, a second polymer of same or different properties may be supplied and the temperature, pressure, tumbling speed and time are set at different levels to achieve, for example, a different thickness of this layer. Similarly, a third polymer may be supplied, and so on.

The application of the above disclosed processes, methods, and systems, is not limited to medicine, pharmaceutical industries. They can be also used in others, such as the biotech, cosmetic and nutraceutical industries.

It may be advantageous to set forth definitions of certain words and phrases used in this patent document. The terms “include” and “comprise,” as well as derivatives thereof, mean inclusion without limitation. The term “or” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like.

As used in this application, “plurality” means two or more. A “set” of items may include one or more of such items. Whether in the written description or the claims, the terms “comprising,” “including,” “carrying,” “having,” “containing,” “involving,” and the like are to be understood to be open-ended, i.e., to mean including but not limited to. Only the transitional phrases “consisting of” and “consisting essentially of,” respectively, are closed or semi-closed transitional phrases with respect to claims. Use of ordinal terms such as “first,” “second,” “third,” etc., in the claims to modify a claim element does not by itself connote any priority, precedence or order of one claim element over another or the temporal order in which acts of a method are performed. These terms are used merely as labels to distinguish one claim element having a certain name from another element having a same name (but for use of the ordinal term) to distinguish the claim elements. As used in this application, “and/or” means that the listed items are alternatives, but the alternatives also include any combination of the listed items.

Throughout this description, the embodiments and examples shown should be considered as exemplars, rather than limitations on the apparatus and procedures disclosed or claimed. Although many of the examples involve specific combinations of method acts or system elements, it should be understood that those acts and those elements may be combined in other ways to accomplish the same objectives.

With regard to flowcharts, additional and fewer steps may be taken, and the steps as shown may be combined or further refined to achieve the described methods. Acts, elements and features discussed only in connection with one embodiment are not intended to be excluded from a similar role in other embodiments.

One embodiment of the invention may be described as a process which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed. A process may correspond to a method, a program, a procedure, a method of manufacturing or fabrication, etc.

For means-plus-function limitations recited in the claims, if any, the means are not intended to be limited to the means disclosed in this application for performing the recited function, but are intended to cover in scope any means, known now or later developed, for performing the recited function.

The foregoing disclosure of the exemplary embodiments of the present invention has been presented for purposes of illustration and description. It is not intended to be exhaustive or to limit the invention to the precise forms disclosed. Many variations and modifications of the embodiments described herein will be apparent to one of ordinary skill in the art in light of the above disclosure. The scope of the invention is to be defined only by the claims appended hereto, and by their equivalents.

Further, in describing representative embodiments of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

Although specific embodiments have been illustrated and described herein for the purpose of disclosing the preferred embodiments, someone of ordinary skills in the art will easily detect alternate embodiments and/or equivalent variations, which may be capable of achieving the same results, and which may be substituted for the specific embodiments illustrated and described herein without departing from the scope of the invention. Therefore, the scope of this application is intended to cover alternate embodiments and/or equivalent variations of the specific embodiments illustrated and/or described herein. Hence, the scope of the invention is defined by the accompanying claims and their equivalents. Furthermore, each and every claim is incorporated as further disclosure into the specification and the claims are embodiment(s) of the invention.

1-4. (canceled)

5. A mixed dose of a nanosized drug wherein at least one portion of the mixed dose comprises a core nanosized drug encapsulated in at least one layer of a protective material, wherein a first portion of the mixed dose comprises a first non-encapsulated core nanosized drug, a second portion of the mixed dose comprises a second core nanosized drug that is encapsulated in one protective layer, a third portion of the mixed dose comprises a third core nanosized drug that is encapsulated in two protective layers and a fourth portion of the mixed dose comprises a fourth core nanosized drug that is encapsulated in three protective layers, wherein all four core nanosized drugs are the same in each of the four portions, and wherein the concentration of the same core nanosized drug is different in at least one of the four portions.

6. The mixed dose of a nanosized drug from claim 5, wherein at least one of the four portions is different in concentration, wherein at least one of the four core nanosized drugs has a different composition and release time than the other nanosized drugs, such that the mixed dose of a nanosized drug is set to act therapeutically following an expected manifestation schedule of different medical conditions in an expected series thereof associated with a medical procedure.

7. The mixed dose of a nanosized drug from claim 5, wherein the protective material for all protective layers has the same composition.

8. The mixed dose of a nanosized drug from claim 5, wherein the core nanosized drug is in solid state.

9. The mixed dose of a nanosized drug from claim 5, wherein the protective material is a polymer.

10-20. (canceled)

21. The mixed dose of a nanosized drug from claim 6, wherein the expected series of medical conditions and their expected manifestation schedule are associated with a tumor removal surgery and comprise, in any order, bleeding, infection, and the presence of leftover tumor.

22. The mixed dose of a nanosized drug from claim 21, further comprising a nutritional mixed dose of a nanosized nutritional composition, the nutritional mixed dose comprising a plurality of portions, wherein at least one of the plurality of portions of the nutritional mixed dose comprises a core nanosized nutritional composition encapsulated in at least one layer of a protective material, such that the patient is continuously supplied with the nutritional composition during the expected series of medical conditions.

23. A mixed dose of a nanosized drug wherein at least one portion of the mixed dose comprises a core nanosized drug encapsulated in at least one layer of a protective material, wherein at least one member of the group consisting of the thicknesses of the at least one layer of a protective material, the composition of the at least one layer of a protective material and the physical configuration of the at least one layer of a protective material, is not the same, thus causing different core nanosized drug release times, and wherein the at least one layer of a protective material has a meshed, compressed or meshed and compressed physical configuration to increase the release time of the core nanosized drug.

24. The mixed dose of a nanosized drug from claim 23 wherein the at least one layer of a protective material has a compressed physical configuration to increase the release time of the core nanosized drug.
